(19) United States
(12) Patent Application Publication
(10) Pub. No.: US 2026/0137427 A1
(43) Pub. Date: May 21, 2026

(54) SYSTEMS AND METHODS FOR PREVENTION OF SET SCREW LOOSENING

(71) Applicant: Nexus Spine, LLC, Salt Lake City, UT (US)

(72) Inventors: David T. Hawkes, Salt Lake City, UT (US); Peter A. Halverson, Salt Lake City, UT (US)

(21) Appl. No.: 19/359,523

(22) Filed: Oct. 15, 2025

Related U.S. Application Data
(60) Provisional application No. 63/708,593, filed on Oct. 17, 2024.

(51) Int. Cl. A61B 17/70

(52) U.S. Cl. CPC A61B 17/7032; A61B 17/7002; A61B 17/7037

(57) ABSTRACT

Implementations of the disclosed systems and methods include medical implants, such as spinal implants for posterior fixation. According to some implementations, the implant includes a tulip configured to receive a rod, along with a set screw configured to fix the rod in place within the tulip. In some cases, the set screw is configured to selectively rotate in a first direction to tighten and to selectively counter-rotate in a second direction to loosen. According to some implementations, the implant includes a mechanical stop configured to prevent undesirable counter-rotation of the set screw to thereby resist loosening of the set screw. Other implementations are discussed.

### RELATED APPLICATION AND PRIORITY CLAIM

This application claims priority to U.S. Provisional Application No. 63/708,593, entitled “SYSTEMS AND METHODS FOR PREVENTION OF SET SCREW LOOSENING” (Attorney Docket No. 23845.192), which was filed on Oct. 17, 2024; the entire disclosure of which is incorporated herein by reference.

### FIELD

The present disclosure relates to medical devices, and more particularly to medical devices having one or more set screws, such as spinal implants.

### BACKGROUND

Medical implants are not infallible. Indeed, medical implants sometimes fail, which can potentially cause pain or damage to a patient's body, and can even put a patient at risk of increased harm or even death. Set screws, which are used in some implants (such as certain spinal implants), have traditionally been one potential point of failure. In particular, traditional set screws often have a tendency to loosen, which can cause failure of the implant as a whole.

Thus, while techniques currently exist that are used to provide implants, challenges still exist, including those listed above. Accordingly, it would be an improvement in the art to augment or even replace current techniques with other techniques.

### SUMMARY

Systems and methods for resisting (e.g., preventing in normal physiological conditions) set screw loosening are provided. For example, some implementations include one or more set screws configured for use in posterior fixation systems (such as those used to secure spinal rods within tulip-style spinal assemblies), where such set screws are configured not to unintentionally counter-rotate or come free of the spinal implants after they have been installed.

While some ordinary set screws have a relatively constant potential energy curve (such that as the screws are loosened, less energy is required to loosen them further), some implementations of the set screw according to the present systems and methods are configured to have a potential energy curve with a local energy minimum, such that at a certain point, more energy is required to loosen the set screw than to tighten it further.

In some implementations, the set screw includes one or more mechanical stops configured to provide the potential energy minimum discussed in the previous paragraph. The mechanical stop can have any suitable form, but in some implementations, it includes one or more processes, tabs, pins, caps, wires, pawls, ratchets, retaining rings, clamps, collars, clips, lock plates, detents, latches, cams, cam followers, dowel stops, indexing plungers, shoulders, abutments, keyways, splines, splined shafts, rails, springs, tangs, lugs, bosses, brackets, or other mechanical stops.

Where the mechanical stop includes one or more processes, any suitable processes can be used. That said, some implementations include one or more bumps, protrusions, bulges, ridges, bosses, tabs, lugs, spurs, flanges, nibs, studs, ribs, bossed pads, tongues, catches, hooks, or other processes configured to catch on one or more complementary stop surfaces (e.g., on the tulip, the rod, or another portion of the implant). In some cases, the process is configured to engage with the complementary stop surface when (and in some cases, only when) the set screw is fully seated in its seat (or is otherwise tightened to a desirable degree).

Where the mechanical stop includes one or more tabs, the tab can include any component configured to be selectively toggled from a first configuration (in which the tab does not act as a mechanical stop) to a second configuration (in which the tab engages with a complementary stop surface to prevent counter-rotation of the set screw). Some implementations of the tab include one or more levers, switches, tags, tabs, arms, flaps, fins, latches, catches, detents or other components configured to switch, pivot, flip, flex, bend, deflect, rotate, slide, rotate, articulate, translate, cam, depress, or otherwise transition from a non-engaged (or disengaged) position to an engaged (or locked) position. In some cases, the tab is configured to plastically deform (e.g., be pressed down or otherwise mechanically deformed) to form the mechanical stop.

Where the mechanical stop includes one or more pins, the pins can include any components configured to be selectively inserted (or otherwise installed) and (in some cases) removed from the set screw to form a mechanical stop. For example, in some cases, the set screw includes one or more passages configured to receive a pin once the set screw is fully seated (which, again, may include being seated to a desired position). The pin can include any suitable type of pin in any size, shape, or configuration, but in some implementations, it is configured to have one or more retaining members (e.g., to keep the pin coupled to the set screw) and one or more stopping members (to engage one or more complementary stop surfaces so the pin can act as a mechanical stop). As with other implementations of the mechanical stop, implementations of the pin can be configured to provide a bi-directional stop (preventing further tightening or loosening of the set screw) or a uni-directional stop (preventing loosening of the set screw, while simultaneously allowing for tightening.

Where the mechanical stop includes one or more caps, the cap can include any feature configured to engage with one or more upper (or any other suitable) portions of the tulip (or other upper surface of the set screw seat). For example, some implementations of the cap are configured to (partially or completely) cover the top of the tulip. In some iterations, a complementary stop surface of the implant is configured to couple to or otherwise interface with the cap.

In some cases, the systems and methods described herein may be particularly suited to spinal fixation constructs (e.g., pedicle, lateral mass, sacral, iliac, cervical, thoracolumbar, or other spinal fixation constructs), particularly those having set screws configured to fix a rod within a tulip.

Some implementations include one or more additional features configured to increase the energy required to loosen the set screw, or other features configured to provide a better surgical experience or better patient outcomes. Thus, these and other implementations are described in greater detail below.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a perspective view of a spinal implant, in accordance with some embodiments of the disclosed systems and methods;

FIG. 2A shows a schematic representing a screw having a threading substantially in the form of an inclined plane, in accordance with some embodiments;

FIG. 2B shows a diagram representing physics of an inclined plane, in accordance with some embodiments;

FIG. 2C shows a graph illustrating the amount of torque (or energy) required to loosen various types of screws as tightness increases, in accordance with some embodiments;

FIG. 2D shows a graph illustrating the amount of torque (or energy) required to loosen a screw configured to have a local potential energy minimum, in accordance with some embodiments;

FIG. 3A shows a perspective view of a set screw, in accordance with some embodiments;

FIG. 3B shows a plan view of a set screw, in accordance with some embodiments;

FIG. 3C shows a perspective view of a spinal implant having a set screw, in accordance with some embodiments;

FIG. 4 shows an elevation view of a set screw, in accordance with some embodiments;

FIG. 5A shows a lower perspective view of a set screw, in accordance with some embodiments;

FIG. 5B shows an elevation view of a set screw, in accordance with some embodiments;

FIG. 6 shows a perspective view of an implant having a set screw, in accordance with some embodiments;

FIG. 7 shows a perspective view of an implant having a set screw, in accordance with some embodiments;

FIG. 8A shows a plan view of an implant having a set screw, in accordance with some embodiments;

FIG. 8B shows an upper perspective view pin of a set screw, in accordance with some embodiments;

FIG. 8C shows a perspective view of a set screw having a pin, in accordance with some embodiments;

FIG. 8D shows a closeup of an interaction of a pin of a set screw with another portion of a spinal implant, in accordance with some embodiments;

FIG. 9A shows a plan view of a pin of a set screw, in accordance with some embodiments;

FIG. 9B shows a perspective view of an implant having a set screw with a pin, in accordance with some embodiments;

FIG. 10 shows a perspective view of an implant having a set screw with a pin, in accordance with some embodiments;

FIG. 11 shows a perspective view of an implant having a set screw, in accordance with some embodiments;

FIG. 12A shows a perspective view of an implant having a set screw with a cap, in accordance with some embodiments;

FIG. 12B shows a perspective view of an implant having a set screw with a cap in accordance with some embodiments, the set screw being rendered transparently to allow viewing of additional details of the implant;

FIG. 12C shows a lower, perspective, cutaway view of a cap of a set screw and an associated portion of an implant, in accordance with some embodiments;

FIG. 12D shows a lower, perspective, cutaway view of a set screw and an associated portion of an implant, in accordance with some embodiments;

FIG. 13 shows an upper perspective view of a portion of an implant, in accordance with some embodiments;

FIG. 14A shows a perspective view of an implant having a multi-part set screw, in accordance with some embodiments;

FIG. 14B shows an exploded view of a multi-part set screw, in accordance with some embodiments;

FIG. 14C shows an assembled multi-part set screw beside a corresponding portion of an implant, in accordance with some embodiments; and

FIG. 14D shows an interaction of a multi-part set screw with a corresponding portion of an implant (extended for clarity), with magnified portions to more clearly show certain details, in accordance with some embodiments.

### DETAILED DESCRIPTION

A description of embodiments will now be given with reference to the figures. It is expected that the present systems and methods may take many other forms and shapes. Hence, the following disclosure is intended to be illustrative and not limiting, and the scope of the disclosure should be determined by reference to the appended claims.

Various reviews of reported failures for posterior fixation and other similar implants indicate that a high percentage (e.g., up to as much as 45%) of reported failures are due to set screws (aka locking nuts/caps). In many cases, the majority of these failures are attributed to set screw loosening.

The causes of set screw loosening may be varied. For example, in some cases, set screws may loosen due to lack of normalization of certain portions of the implant. In some cases, the polyaxial limit may be exceeded when the implant is installed. In some cases, the system may experience a reduced locking force due to reduction. Furthermore, movement of a patient (as is typically experienced post-operatively) can lead to vibration, a variety of forces from different directions, and spontaneous loosening. Embodiments of the present systems and methods are configured to address these and other problems.

As an example of a system that may be used in connection with some embodiments of the described systems and methods, some embodiments include or are used in connection with one or more spinal implants 20. The described systems can be used with any suitable spinal implant (e.g., one or more pedicle screw systems, rod-to-screw connectors, lateral mass screws, sacral screws, cervical fixation systems, occipitocervical constructs, thoracolumbar constructs, interbody fusion devices, expandable cages, or other suitable spinal implants). Although any suitable spinal implant can benefit from embodiments of the described systems and methods, in some cases, however, a spinal implant configured for posterior fixation is used.

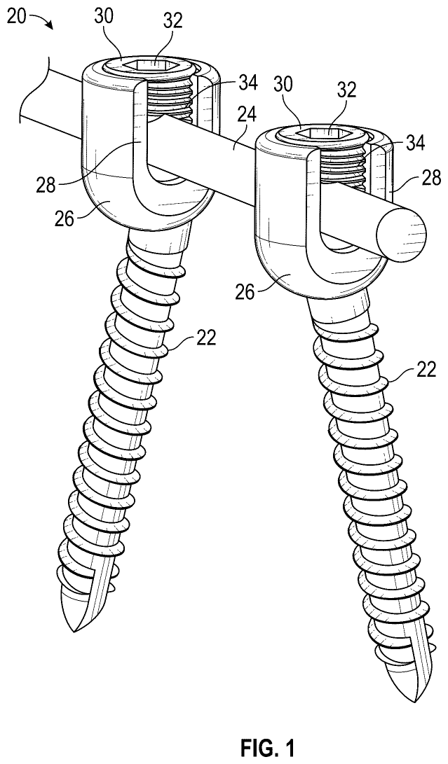

In some embodiments, the described spinal implant 20 includes one or more anchors 22, such as pedicle screws or any other suitable anchors (e.g., pedicle screws, other screws, nails, bolts, pins, rivets, ties, threaded engagements, or other components configured to anchor one or more portions of a spinal implant to one or more bones, tissues, sinews, or other parts of a patient's body or hardware installed within the patient's body). By way of non-limiting illustration, FIG. 1 shows a spinal implant 20 having two anchors 22 in the form of pedicle screws that are configured to be inserted into adjacent spinal vertebrae.

In some embodiments, the spinal implant 20 includes one or more rods 24. The rod can include any suitable component for helping to align a portion of the patient's spine (or any other suitable portion of the patient), to otherwise provide a certain structure or function (e.g., correcting scoliosis, lordosis, kyphosis, or other spinal deformities, helping to carry out spinal fusion, or otherwise), or to serve any other suitable purpose. The rod can also have any suitable shape (although it typically includes an elongated component, which may form one or more lines, curves, angles, or other shapes as may be useful for spinal surgery). It can also have any suitable cross-section (e.g., circular, semi-circular, elliptical, ovular, oval-shaped, triangular, square, rectangular, trapezoidal, pentagonal, hexagonal, star-shaped, T-shaped, polygonal, or any other regular or irregular shape). By way of non-limiting illustration, FIG. 1 shows a cylindrical spinal rod 24 coupled to multiple pedicle screw anchors 22.

In some embodiments, the spinal implant 20 includes one or more tulips 26. The tulip can include any suitable component for coupling the anchor 22 to the rod 24. Some embodiments of the tulip include one or more traditional posterior fixation tulips (e.g., having a bottom with an opening formed therein for a pedicle screw, a receptacle configured to receive the rod, and a top having threading configured to receive one or more set screws 30 to prevent the rod from coming out of the receptacle through the top). However, the tulip can include any other suitable component configured to couple the rod to the anchor. For example, some embodiments include one or more implant bodies configured to operate as a tulip and rod together, or any other alternative implant component configured to receive a set screw.

As mentioned above, some embodiments include one or more set screws 30. While such set screws can include traditional set screws (e.g., short, generally cylindrical, generally flat screws configured to lock a rod 24 within a tulip 26), they can include any suitable feature allowing them to perform any suitable locking function (e.g., to lock one or more components together, hold one or more components in place, or otherwise “set” one or more components of an implant, such as a rod of a spinal implant 20).

The set screw 30 can have any suitable dimensions. For example, some embodiments are sized and configured to fit into the top of a tulip 26, while others are sized and configured to be used with other implant components.

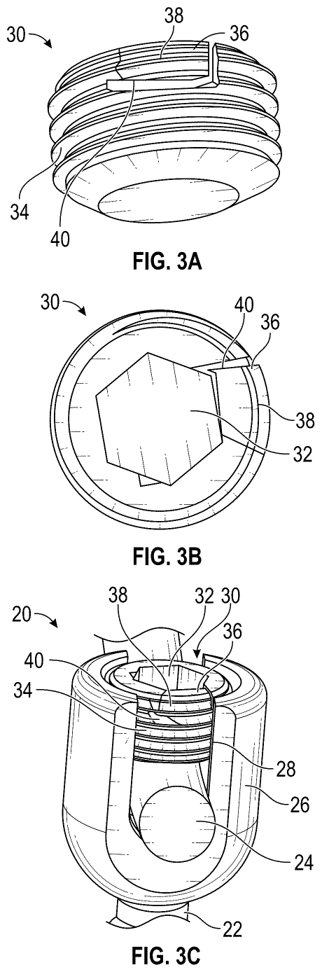

The set screw 30 can have any suitable shape, including being cylindrical, disc-shaped, having a disc-shaped portion or a threaded portion with a process extending therefrom, or having any other suitable shape. In some embodiments (e.g., as illustrated in FIGS. 1 and 3A), the set screw 30 is substantially cylindrical.

The set screw 30 can have any suitable feature allowing it to perform its intended function (as discussed above). For example, some embodiments include one or more drive features 32. In this regard, the drive feature can include any suitable process, recess, or other interface allowing the set screw to interface with a drive or similar component of a tool. For example, some embodiments include a drive feature that has one or more of the following configurations: slotted, Phillips, phil-slot, hex socket, spline socket, star, torx, Tor-Set™, tri-wing, posidrive, timmit, square, combo-square, Frearson, 6-lobe, 12-point, spanner, clutch, one-way, or any other drive configuration. By way of non-limiting illustration, FIG. 1 shows a spinal implant 20 with a set screw 30 having a drive feature 32 configured to receive a star-drive bit of a tool.

Some embodiments of the set screw 30 include one or more sections of threading 34. The threading can include any suitable threading that allows the set screw to be twisted into complementary threading (e.g., formed in a tulip or any other suitable portion of an implant). The threading can include any suitable: size threading, crest, flank, root, major diameter, minor diameter, nominal size of thread, shape, size, pitch, threads per centimeter or per inch, lead, thread angle, cross-sectional shape, fit class, allowance, tolerance, thread hand, number of starts, surface-related characteristic, coating, finish, pitch diameter, lead angle, thread depth, flank angle, helix angle, pitch diameter, engagement length, reverse-angle thread, variable pitch thread, interrupted thread, or other characteristics that may be desirable to allow the set screw to interface with a complementary threading. In some embodiments, the threading is configured such that rotating the set screw in a first direction (e.g., clockwise), causes the set screw to tighten, whereas counter-rotating the set screw in a second direction (e.g., counterclockwise) causes the set screw to loosen.

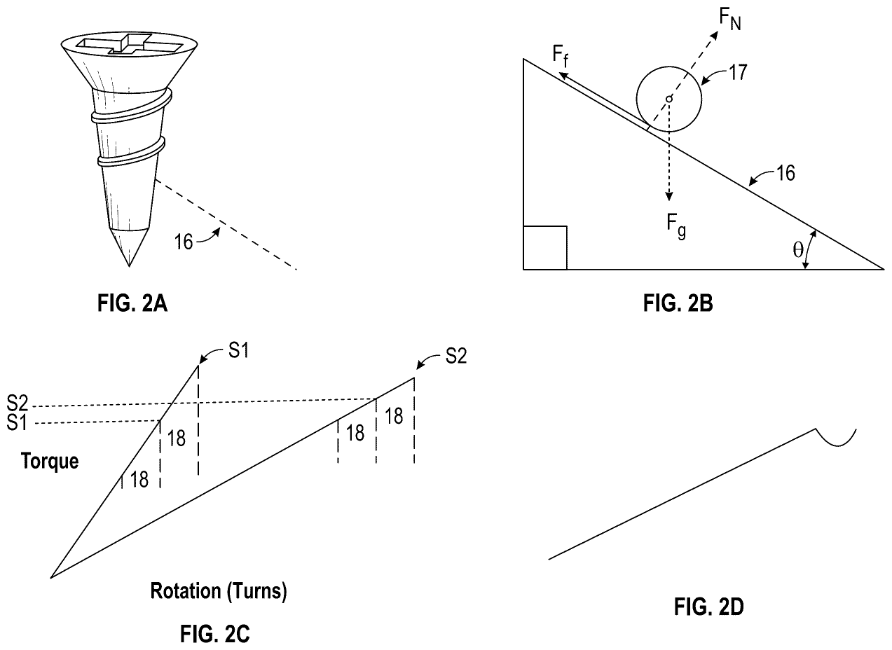

Generally speaking, from a simplified mechanical perspective, a threading 34 of a screw can be conceptualized as an inclined plane 16 wrapped around a cylinder, as shown in FIG. 2A. As with any inclined plane, the threading includes a higher energy state and a lower energy state. This can be conceptualized by picturing a ball 17 on an inclined plane 16 (as shown in FIG. 2B). Wherever the ball is located on the inclined plane, the ball will naturally (under the pull of gravity) move from a higher location on the plane (a higher energy state) to a lower location on the plane (a lower energy state). In the case of some ordinary screws, a tight screw is at a higher energy state than is a loose screw. Because of this, the screw will naturally loosen over time. Any natural movement, vibrations, forces from various directions, or other disturbances of sufficiently high energy can cause or allow the screw to rotate in a net loosening direction.

In some cases, loosening of the set screw 30 can be resisted by increasing the amount of energy required to rotate the screw (e.g., by making the screw have a tighter fit; by using one or more deformable elements to reduce the decrease in preload, thereby increasing the rotation needed to decrease the force necessary for loosening; increasing an amount of force necessary to loosen the screw to an amount that exceeds the breaking point of the screw; or in any other suitable manner). By pursuing this course of action, the effect is that shown in the graph in FIG. 2C. In particular, a lower energy screw (S1) and a higher energy screw (S2) have a similar energy curve: as each set screw is loosened, less energy is required to loosen it further. When S2 counter-rotates a quarter turn 18, it requires a higher torque to rotate another quarter turn 18 than S1. Thus, more work (the area under each curve in FIG. 2C) is required to loosen S2 than S1. However, this does not necessarily create a local potential energy minimum, so the tendency in both cases is still to loosen over time—even if S2 takes more energy to loosen than does S1.

In contrast, some embodiments of the described systems are configured such that one or more localized minima are created in the potential energy curve to resist (e.g., decrease the incidence of, decrease the magnitude of, increase the energy required to carry out, prevent, or otherwise resist) counter-rotation of the set screw and thereby resist set-screw loosening. By way of non-limiting illustration, FIG. 2D shows an energy curve with a local minimum. Thus, instead of having a tendency to always loosen (no matter how tight the screw), once tightened, some embodiments with a local minimum have a tendency to always move toward the minimum (which is still in a tightened configuration)—thus removing the tendency to loosen over time.

The local minimum can be created in any suitable manner (e.g., via elastic interference, plastic interferences, ratchet geometry, frictional engagements, mechanical engagements, or in any other suitable manner). In some embodiments, the set screw 30 includes one or more mechanical stops 36 (of the same or different types) configured to create a local energy minimum in the energy curve. For example, some embodiments include one or more processes, tabs, pins, caps, wires, pawls, ratchets, retaining rings, clamps, collars, clips, lock plates, detents, latches, cams, cam followers, dowel stops, indexing plungers, shoulders, abutments, keyways, splines, splined shafts, rails, springs, tangs, lugs, bosses, brackets, recesses, hooks, latches, irregularities, tongue-and-groove connections, or other types of mechanical stops configured to physically interfere with counter-rotation.

Moreover, the set screw 30 can have any suitable number of mechanical stops. Some embodiments have one, two, three, four, five, six, seven, eight, or more mechanical stops (of the same or any different combination of types of mechanical stops as described herein).

According to some embodiments, the mechanical stop 36 includes one or more processes 38. The processes can include any suitable protrusions configured to physically interfere with counter-rotation, such as one or more bumps, ridges, bulges, tags, flanges, catches, protuberances, corrugations, ribs, bosses, tabs, lugs, spurs, flanges, nibs, studs, ribs, bossed pads, barbs, or other processes.

In some embodiments, one or more processes 38 is formed from one or more portions of a set screw body, modified to protrude and provide a physical interference against counter-rotation. In this regard, some embodiments include one or more slots 40 cut into a body of the set screw 30 to allow for a portion of the set screw body to protrude away from other portions of the set screw body (e.g., as a resilient flange). In such embodiments, the slot can include any suitable slot, such as one or more cuts, channels, holes, grooves, apertures, or other slots that allow for a portion of the set screw 30 to be bent or flexed outward, or to otherwise create a mechanical stop 36. By way of non-limiting illustration, FIGS. 3A-4 show various slots 40 cut into set screws 30 to create one or more processes 38 in the form of a protruding portion of the body of the set screw 30. Although such a process can have any suitable characteristic, in some embodiments it is slightly bent so as to be biased away from a central or longitudinal axis of the set screw so as to be configured to increase pressure between the process and corresponding threading (e.g., of the tulip 26).

Although the slot 40 can be formed in any suitable manner to allow for the process 38 to be created, some embodiments of the slot include one or more directional cuts along different axes of the set screw 30. For example, some embodiments include one or more cuts along (or generally along, such as within (or less than) 5 degrees, 10 degrees, 20 degrees, 30 degrees, 45 degrees, or 90 degrees (or within any subrange thereof) of) a central or longitudinal axis of the set screw, and one or more cuts along (or generally along) a circumference of the set screw. In some embodiments, the horizontal cut aligns with (e.g., is parallel, substantially parallel, or within 15 degrees of being parallel to) a portion of the threading 34, whereas in some embodiments, the horizontal cut runs across (e.g., is not parallel to) the threading. Thus, the process can have any suitable shape and can run at any angle or in any manner that allows it to function as described herein.

By way of non-limiting illustration, FIGS. 3A-3C show some embodiments of set screws 30 having a slot 40 that include a central axis cut that is contiguous with a circumferential cut (which runs across the threading), thereby forming a generally L-shaped slot to allow for a portion of the set screw body (e.g., a portion at the top) to form a resilient or biased process 38 to act as a mechanical stop 36. FIG. 4 shows a set screw 30 having a slot 40 that includes a central axis cut 40a joining two circumferential slots 40b (which run along the threading 34), thereby forming a generally U-shaped slot to allow for a portion of the set screw body (e.g., a portion near the middle) to form a process 38 to act as a mechanical stop 36.

The slot 40 can be in any suitable location. In some embodiments, it is at or near the top (e.g., as shown in FIGS. 3A-3C), in some embodiments it is at or near the middle (e.g., as shown in FIG. 4), and in some embodiments it is at or near the bottom of the set screw 30. It can also be in any other suitable location.

In some embodiments, the slot 40 is configured to allow for at least a portion of the process 38 to be pushed back in (manually or otherwise, e.g., towards a central axis of the set screw 30) to selectively allow for counter-rotation of the set screw. For example, an uncut portion of the set screw body may act as a hinge to allow the process to push inward or protrude outward. In some embodiments, the drive feature 32 (e.g., a recess that is configured to receiving a driving tool) is deep enough that the slot is cut clear through a wall of the set screw body from an exterior to an interior (inside the drive feature). In some embodiments, at least a portion (or in some cases, all) of the set screw is hollow to allow for the slot to be cut through to the hollow portion.

The slot 40 can occupy anywhere between 1 degree and 270 degrees of the circumference, or any subrange thereof (such as between 10 degrees and 120 degrees, 15 degrees and 45 degrees, or any other subrange). In some cases, the groove is sized to optimally provide a mechanical stop 36 while avoiding overly weakening the set screw 30.

In some embodiments (and as mentioned above), the process 38 is biased outward so it forms a mechanical stop to prevent counter-rotation by default. In this regard, the process can be biased in any suitable manner, including by being bent, flexed, pushed with a spring or other resilient component, comprising a resilient material, being resiliently hinged, or otherwise biased). By way of non-limiting illustration FIG. 3B shows an embodiment in which the process 38 is slightly bent so as to resiliently protrude (when at rest) from a circumference of the set screw 30.

According to some embodiments, the mechanical stop 36 is configured to interact with one or more complementary stop surfaces. In such embodiments, the complementary stop surface can include any suitable component that allows the set screw 30 to selectively be locked in position (e.g., a catch, a recess, a barb, a flange, a tang, a shoulder, an orifice, a portion of the implant 20, any other suitable complimentary stop surface). In some cases, however, the complementary stop surface comprises a portion of the implant 20, such as an edge 28 of the tulip 26 or any other suitable portion. By way of non-limiting illustration, FIG. 3C shows an embodiment depicting how a mechanical stop 36 in the form of a process 38 interacts with a complementary stop surface in the form of an edge 28 of a tulip 26, thereby preventing counter-rotation unless the process 38 is pressed inward (e.g., by a practitioner) to allow for the set screw 30 to be removed or loosened.

According to some embodiments, the mechanical stop 36 includes a process 38 in the form of a bump or other protrusion. In some embodiments, the process is configured to be coupled to the set screw 30, but in some embodiments, the process is integrally formed with the set screw (e.g., a portion of the material of the set screw body is configured to bulge or otherwise protrude outward to form the process). The process can be formed on any suitable part of the set screw. For example, in some embodiments, the process is formed on a bottom of the set screw. In other embodiments, the process is formed on a side, a top, or any other suitable location. Where the bump is on the bottom of the set screw, it can be in any suitable position (e.g., in the center, on the perimeter, between the center and the perimeter, or at any other location), but in some cases it is positioned near a perimeter such that it has substantial circumferential motion as the set screw rotates.

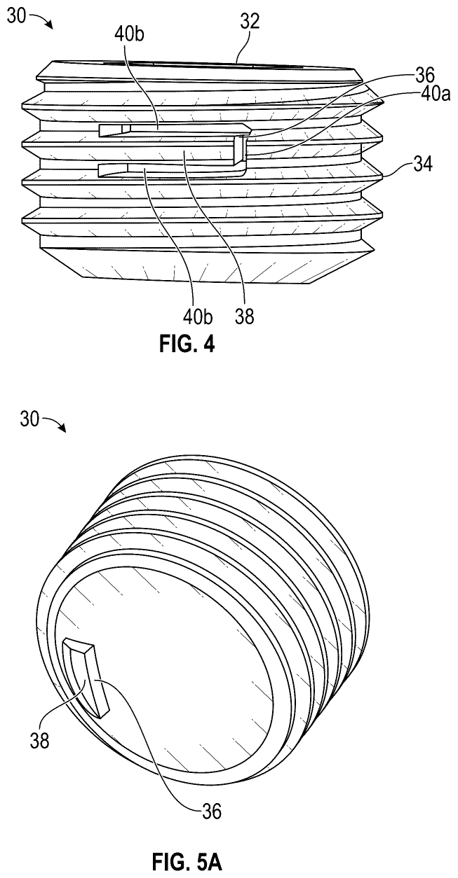

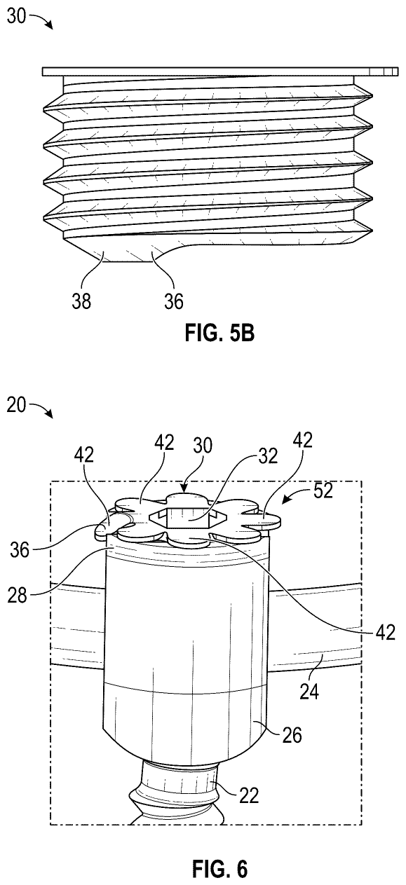

In some embodiments, the process 38 is formed as a bump on the bottom of the set screw 30 and is configured to create a mechanical stop 36 against a corresponding stop surface on a portion of the rod 24 (or a portion of the tulip 26 or any other suitable portion of the spinal implant 20). In some cases, while the screw is not yet fully seated, the bump is elevated from the corresponding stop surface (as the bottom of the set screw is elevated), yet when the screw reaches its fully seated (or other desired) position, the bump lowers into place, sliding right into a position where undesirable counter-rotation is prevented by physical interference of the bump against a corresponding stop surface. By way of non-limiting illustration, FIGS. 5A-5B show a set screw 30 having a mechanical stop 36 in the form of a process 38 in the form of a bump on the bottom of the set screw, the bump positioned with respect to the threading such that it engages with a corresponding stop surface on an implant (e.g., a rod, a portion of a tulip, or any other suitable surface) when the set screw is fully seated.

Where the set screw 30 includes the bump, the bump can have any suitable shape allowing it to act as a mechanical stop 36. For example, some embodiments of the bump are generally circular, semi-circular, triangular, square, rectangular, trapezoidal, pentagonal, hexagonal, star-shaped, T-shaped, S-shaped, lobed, polygonal, teardrop shaped, comma shaped, tapered, sloped, or any other regular or irregular shape that allows the bump to function as a mechanical stop. That said, some embodiments are generally rectangular with one or more chamfered, sloped, or beveled edges to allow for uni-directional rotation past a corresponding stop surface, or for selective bi-directional rotation past a corresponding stop surface. Where selective bi-directional rotation is allowed, the set screw can be configured such that counter-rotation only occurs at forces well beyond those experiences within the human body without purposeful intervention.

According to some embodiments, the mechanical stop 36 includes one or more tabs 42. The tab can include any component having at least a first position (or configuration) and a second position (or configuration), where the tab allows the set screw 30 to freely rotate bi-directionally (or in at least one direction) while the tab is in the first position (e.g., the tab does not act as a mechanical stop for at least one direction of rotation of the screw when the tab is in the first position), but the tab acts as a mechanical stop when it is in the second position (preventing at least counter-rotation, and in some cases preventing rotation bi-directionally, such as to prevent loosening and over-tightening simultaneously). By way of non-limiting illustration, FIG. 6 shows an implant 20 having a set screw 30 with a mechanical stop 36 in the form of a plurality of tabs 42 (integrated into a cap 52, as discussed in more detail below), with each tab being configured to be plastically deformed to form a mechanical stop that interfaces with any suitable corresponding stop (e.g., in the form of an edge 28 of a tulip 26).

Where the mechanical stop 36 includes one or more tabs 42, any number of tabs can be included. For example, some embodiments include only a single tab, whereas some embodiments include two, three, four, five, six, seven, eight, nine, ten, or more tabs. By way of non-limiting illustration, FIG. 6 shows an embodiment comprising eight tabs 42.

The tabs 42 can be in any suitable location or position to allow them to be toggled or otherwise moved between the first position and the second position. For example, in some embodiments, the tabs are positioned around a perimeter of a portion of the set screw 30, such that one or more different tabs can be engaged depending on when and where the set screw is fully seated or otherwise reaches a desirable position. By way of non-limiting illustration, FIG. 6 shows an embodiment in which a plurality of tabs 42 are positioned around a perimeter of a set screw 30 for easy deployment.

As with the other types of mechanical stops 36, the tab 42 can be in any desirable position, such as on the top, on the side, on the bottom, or even on the interior of the set screw, as long as the tab can be toggled from the first (non-interfering) position to the second (mechanical-stop-active) position. By way of non-limiting illustration, FIG. 6 shows an embodiment in which the tabs 42 are disposed at a top, lateral portion of the set screw 30.

While some embodiments of the tab 42 are configured to be plastically deformed to toggle from the first position to the second position, some embodiments are configured to toggle in other ways. For example, in some cases, the tab includes one or more switches, buttons, levers, clasps, detents, mechanical mechanisms, hinges, catches, or other toggles (thus, some embodiments do not use plastic deformation). In this regard, while the tab can be moved or otherwise toggled in any suitable manner, some embodiments of the tab are configured to be toggled via switching, turning, rotating, plastically deforming, sliding, flipping, elevating, pressing, pulling, or otherwise changing the position or shape of one or more components.

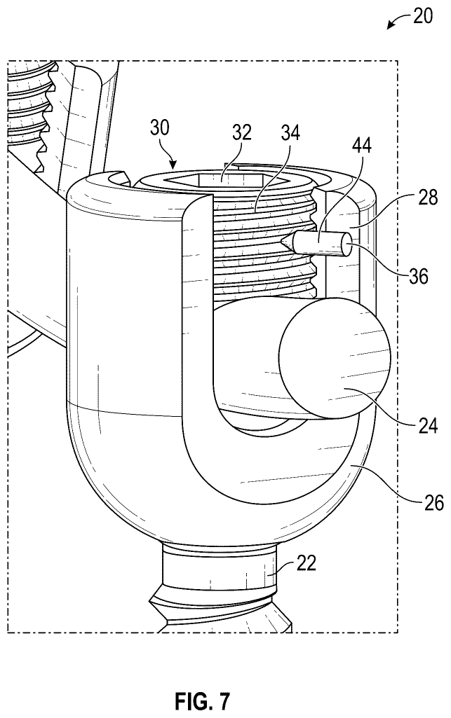

According to some embodiments, the mechanical stop 36 includes one or more pins 44. The pin can include any component configured to be selectively installed or actuated to act as a mechanical stop (and, in some cases, selectively removed or reverse actuated as well). In some embodiments, when the pin is not installed or is not fully actuated, the set screw 30 is allowed to freely rotate bi-directionally, but when the pin is in place, rotation is limited (at least uni-directionally, but in some cases, bi-directionally). For example, the pin can include any pin, clasp, catch, pinion, prong, shim, cotter pin, clevis pin, dowel pin, spring pin, lynch pin, taper pin, hitch pin, wire lock pin, detent pin, quick release pin, handle pin, cover pin, ballpoint pin, grooved pin, retractable pin, silk pin, knurled pin, safety pin, lapel pin, carabiner, pawl, wire, or any other component configured to be selectively coupled to a set screw to act as a mechanical stop. By way of non-limiting illustration, FIG. 7 shows a set screw 30 having a passage configured to receive a pin 44, wherein when the pin 44 is in place, the pin 44 acts as a mechanical stop 36 that interacts with any suitable corresponding stop surface (e.g., an edge 28 of a tulip 26) to prevent counter-rotation of the set screw 30.

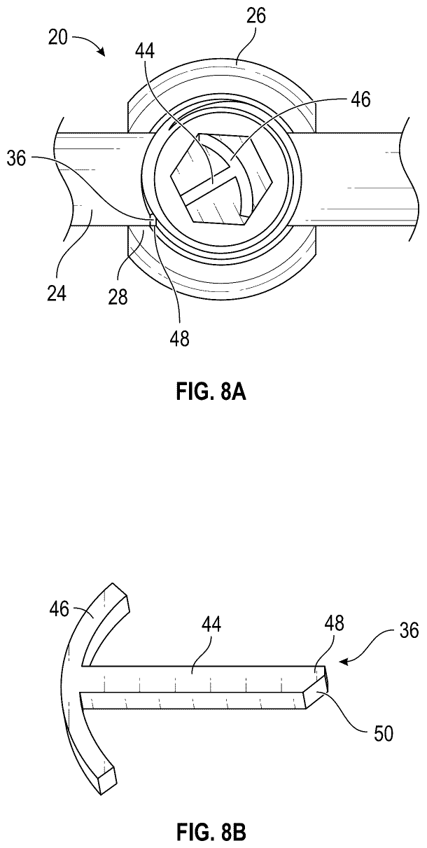

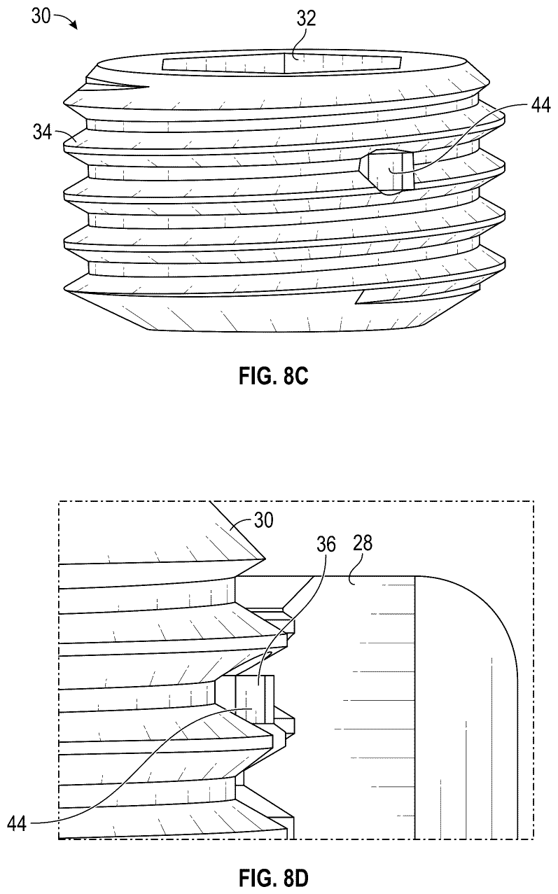

According to some embodiments, the pin 44 includes one or more retaining members 46. The retaining members can include any component suitable for retaining the pin in or on (or otherwise coupling the pin to) the set screw 30. In some cases, the retaining member is configured to insert into or through one or more passages (or recesses, slots, drive features 32, or other receiving features) of the set screw. For example, FIG. 7 shows a set screw 30 with a retaining member 46 of a pin 44 configured to insert into a passage of the set screw 30 (e.g., via the drive feature 32).

Where the set screw 30 includes one or more passages for receiving a pin 44, any suitable number of passages can be used. For example, some embodiments include one, two, three, four, five, six, seven, eight, or any other number of passages formed in the set screw in any suitable manner configured to receive one or more portions of a pin. In some cases, the passages are formed around a circumference of the set screw, such that the pin can be inserted into any suitable passage to selectively lock the set screw in place once the pin is fully seated (or placed in any other suitable desired location). In some cases, one or more passages are configured to properly align when the set screw 30 is fully seated (such that an inserted pin 44 would properly interact with a corresponding stop surface (e.g., an edge 28 of a tulip 26), as shown in FIGS. 7 and 8D). In this manner, some embodiments need only a single passage for receiving a pin, with the passage being disposed in the proper location.

In some embodiments with multiple passages, the passages are offset from each other (horizontally along the circumference of the set screw, vertically, or otherwise). In some cases, using offset passages can alter (e.g., increase) the contact angle or change the deflection of the pin 44, depending on the passage in which the pin is disposed. In some embodiments, a mechanical stop 36 can be efficiently implemented without placing undue stress on the implant 20, as different stresses can be selected by placing the pin through different passages.

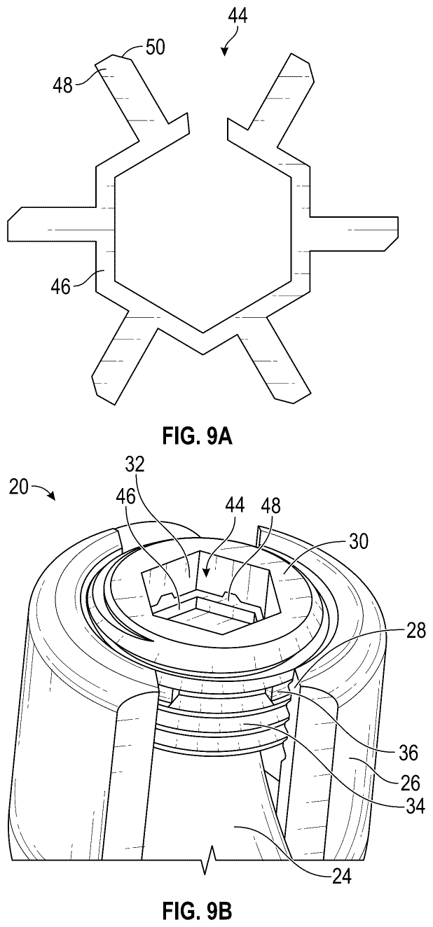

In some embodiments, the retaining member 46 is configured to interact with any suitable portion of the set screw 30 (i.e., a drive feature 32, a top, bottom, side, edge, or any other portion of the set screw. In some embodiments, however, the retaining member is configured to interact with an interior of a drive feature of the set screw. For example, FIG. 8A shows a set screw 30 with a drive feature 32, with a pin 44 having retaining one or more members 46 disposed within to selectively anchor the pin 44 within the drive feature 32.

Where one or more retaining members 46 are configured to retain the pin 44 within the drive feature 32, the retaining members can be configured to do so in any suitable manner. For example, some embodiments of the retaining members are configured to correspond to, contact, or otherwise retain the pin against one or more points, edges, corners, slots, or other portions of the drive feature. By way of non-limiting illustration, FIG. 8A shows an embodiment in which a pin 44 has retaining members 46 that are configured to correspond to two opposing points of a hexagonal drive feature 32. By way of further non-limiting illustration, FIG. 9A shows an embodiment with a pin 44 having a retaining member 46 that generally corresponds to a shape of a drive feature.

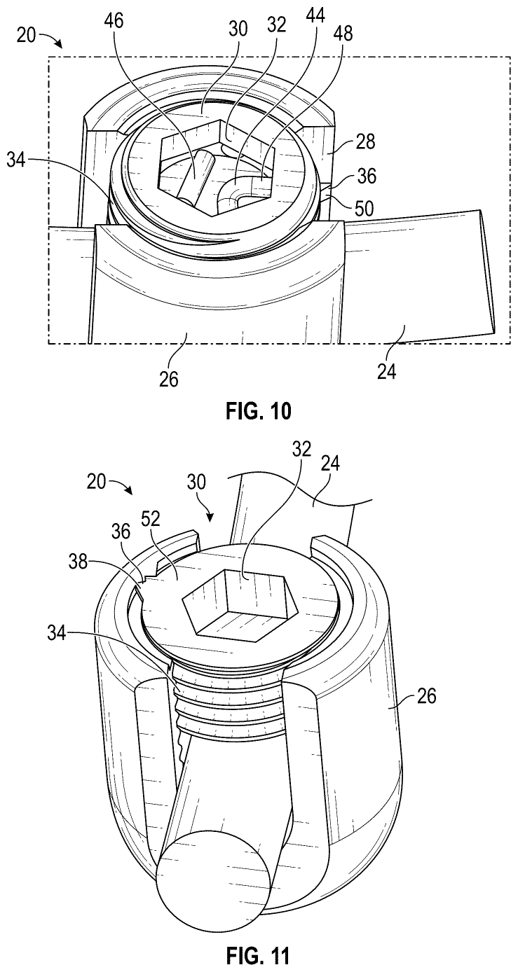

According to some embodiments, the retaining member 46 is configured to selectively retain the pin 44 in the drive feature 32 (or otherwise coupled to the set screw 30). While this can be done in any suitable manner, some embodiments of the retaining member include one or more resilient or bendable components configured to be compressed, bent, or otherwise selectively manipulated to allow for removal from the drive feature or other decoupling from the set screw. For example, some embodiments include one or more curved portions that can be pinched or otherwise flex (e.g., inward or in any other suitable manner) to allow for removal (e.g., as shown in FIGS. 8A-8B). Some embodiments of the retaining members 46 include one or more gaps to allow for compression or contraction to allow for a decoupling (e.g., as shown in FIGS. 9A-9B). Some embodiments include one or more hairpin sections or other components configured to be pinched together or to be deformed to decrease the overall volume the pin occupies to allow for removal (e.g., as shown in FIG. 10). In line with the foregoing, some embodiments of the pin include a first configuration in which the pin is selectively and mechanically prevented from decoupling from the set screw, and a second configuration in which the pin is selectively allowed to decouple from the set screw.

Some embodiments of the pin 44 include one or more stopping members 48. The stopping member can include any portion of any pin configured to act as a mechanical stop 36. In some embodiments, the stopping member includes a portion of the pin configured to protrude from or through a portion of the set screw 30 (e.g., through a passage) and interact with a complementary stop surface external to the set screw. In some embodiments, one or more retaining members 46 of the pin are configured to be disposed interior to the set screw, while one or more stopping members are configured to be disposed exterior to the set screw.

The stopping member 48 can have any suitable shape or configuration, and it can be configured to protrude from or through any portion of the set screw 30. Indeed, some embodiments of the stopping member are configured to have one or more characteristics of a process 38 as discussed herein.

According to some embodiments, the stopping member 48 is configured to act as a mechanical stop bi-directionally. That said, some embodiments of the stopping member are configured to act as a mechanical stop uni-directionally (e.g., allow for tightening of the set screw, while resisting set screw loosening). While this can be done in any suitable manner, some embodiments include one or more rounded, sloped, curved, angled, or other chamfered portions 50, such that a chamfered portion of the stopping member can slide past a corresponding stop surface to not force a stop, but a non-chamfered portion catches on a corresponding stop surface and acts as a mechanical stop 36. By way of non-limiting illustration, stopping members 48 with a uni-directional-rotation chamfered portion 50 are shown in FIGS. 8A-10.

According to some embodiments, the pin 44 comprises one or more wires (e.g., a stiff wire, a spring wire, or another type of wire) or other elongated components, which in some cases are shaped to form the pin. In some cases, one or more reliefs are cut or otherwise formed in the set screw 30 to provide for retention of the elongated component. Where the pin includes a wire, any suitable type of wire may be used. For example, some embodiments utilize cylindrical wire, as this may provide for ease of manufacturing (in some cases).

On the whole, the pin 44 can have any suitable shape (e.g., being anchor-shaped, star-shaped, wheel-and-spokes-shaped, polygonal, regular, irregular, symmetrical, asymmetrical, curved, straight, aligned with internal geometry of the set screw, U-shaped, C-shaped, T-shaped, Y-shaped, X-shaped, S-shaped, P-shaped, or otherwise shaped). By way of non-limiting illustration, FIG. 8B shows an embodiment in which the pin 44 is anchor-shaped.

In accordance with some embodiments, the set screw 30 includes one or more caps 52 configured to act as or include one or more mechanical stops 36. In this regard, the cap can include any component configured to modify a top (or any other portion) of the set screw to provide a mechanical stop. In some embodiments, the cap is configured to couple to the top of the set screw, and in some embodiments, the cap is integrally formed with or otherwise enslaved with the set screw. Where a cap 52 is included, the cap can provide a mechanical stop 36 in any suitable manner. For example, some embodiments of the cap include one or more other mechanical stops as described herein (e.g., one or more processes 38, tabs 42, pins 44, or other mechanical stops). The cap can also include any feature of the top of the set screw (e.g., a drive feature 32). By way of non-limiting illustration, FIG. 11 shows a set screw 30 having a cap 52, which in turn includes a process 38 in the form of a protrusion on a perimeter of the cap 52 configured to engage with a corresponding stop surface (e.g., a recess, catch, barb, or other stop surface of a tulip 26). In this case, the cap 52 is integrally formed with the set screw 30, has a slightly larger diameter than the remainder of the set screw 30, and includes a drive feature 32 (or allows access to the drive feature 32 of the set screw 30).

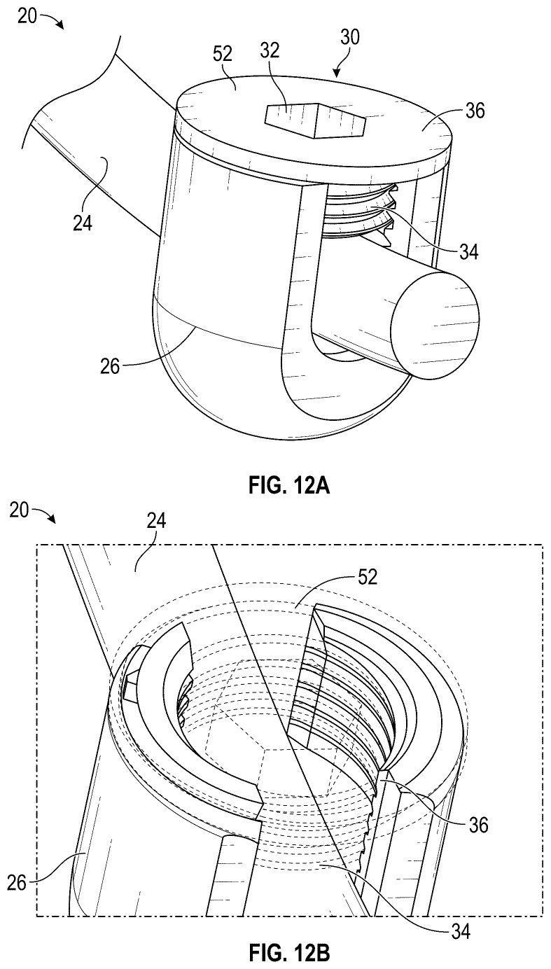

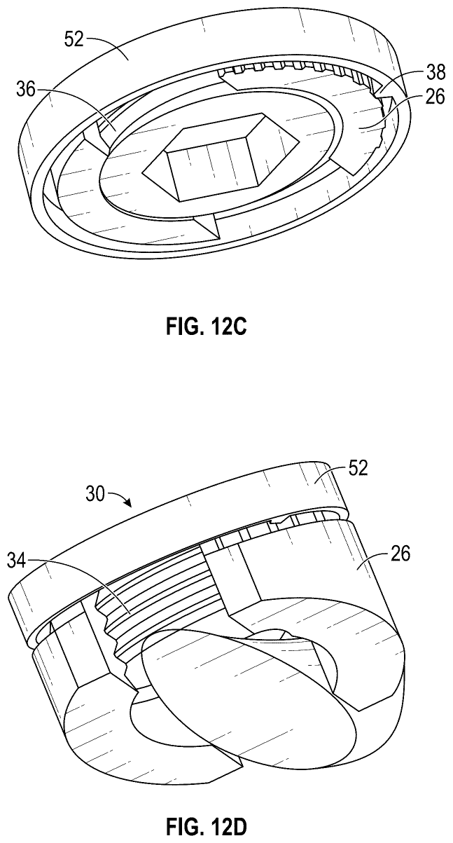

According to some embodiments, the cap 52 is configured to interact with one or more portions of the implant 20 in which the set screw 30 is installed. For example, some embodiments of the cap are configured to interact with a tulip 26. Some embodiments of the cap have a larger diameter than (or a portion that extends outwardly from) a body of the set screw (e.g., the portion having the threading 34). Some embodiments of the cap are configured to cover all or part of the top of the tulip. By way of non-limiting illustration, FIGS. 12A-12D show an embodiment of a set screw 30 with a cap 52 configured to cover a top edge of at least a portion of a tulip 26.

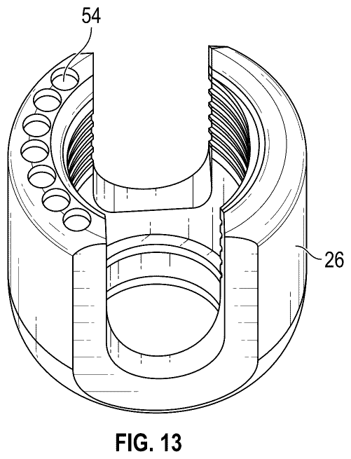

According to some embodiments, the tulip 26 includes one or more stop features 54 configured to interact with the cap 52 when the set screw 30 is fully seated (or otherwise disposed in a desired location). While such stop features can take any shape or form, some embodiments include one or more processes, recesses, tongues, grooves, slots, ridges, catches, flanges, flaps, blades, or other features configured to form an interference fit, tongue-in-groove connection, or other coupling with the cap. By way of non-limiting illustration, FIG. 13 shows a tulip 26 having a plurality of stop features 54 in the form of recessed shapes (e.g., circles) set in a top edge of the tulip 26 into which processes 38 (e.g., beads, bumps, or other processes) of the corresponding cap 52 can fit when the set screw 30 is tightened and the cap 52 rotates into place to cover the top edge of at least a portion of the tulip 26.

The set screw 30 can include any combination or permutation of types of mechanical stops 36, as discussed herein. For example, some embodiments include a first process 38 and a second process (of the same type or a different type than the first process). Some embodiments include a process and a tab 42. Some embodiments include a process and a pin 44. Some embodiments include a process and a cap 52. Some embodiments include a tab and a pin. Some embodiments include a tab and a cap. Some embodiments include a pin and a cap. Some embodiments include a process, a tab, and a pin. Some embodiments include a process, a pin, and a cap. Some embodiments include a process, a tab, and a cap. Some embodiments include a tab, a pin, and a cap. Some embodiments include each of a process, a tab, a pin, and a cap. Some embodiments include multiple of any of processes, tabs, pins, and caps, in some cases with other types of mechanical stops as well. Thus, the set screw is not limited to having only the mechanical stops shown in a single figure.

The mechanical stop 36 can also have any other suitable feature. In some embodiments, the mechanical stop is configured not to activate or engage until the last rotation of the set screw 30 (e.g., the set screw is fully seated within its seat). For example, in some embodiments, the mechanical stop is formed near a top of the set screw, such that if the top is not yet disposed within the seat, the screw can be backed up without engagement of the stop. In some embodiments, even after the stop is engaged, the screw can be backed up by disengaging the stop. That said, in some cases, the mechanical stop is configured such that spontaneous and undesired disengagement is impossible or highly unlikely, such as by requiring a mechanical force to push inward on the mechanical stop or otherwise change it to a disengaging configuration in order to disengage it.

In some embodiments, one or both of the mechanical stop 36 and the corresponding stop surface is on an additional component (e.g., other than the set screw 30, the tulip 26, the implant 20, or the rod 24). For example, in some embodiments, a dedicated stopping member is included, which can then be attached to one or more of the rod, the tulip, the implant, and the set screw. In some cases, the stopping member can include a component (e.g., a blade, barb, protrusion, catch, flange, recess, latch, or any other suitable component) to be inserted between the threading of the set screw and the threading of the set screw seat. Such additional component can include any object configured to be coupled (e.g., welded, adhered, attached, inserted into a passage or hole, or otherwise coupled) to one or more components of the implant 20.

In some embodiments, a set screw 30 is configured to selectively loosen in one direction (e.g., a counterclockwise direction), while a second component (e.g., another set screw or a different component) is configured to apply tension in an opposite direction (e.g., where another set screw is used, the other set screw could loosen in a clockwise direction), and the set screw is coupled to the second component such that the tension or pressure of the second component resists the set screw's loosening (in some cases, this allows two set screws to resist each other's loosening).

According to some embodiments, the set screw 30 includes one or more resistance mechanisms that are configured to increase the force required to loosen the set screw (e.g., instead of or addition to inclusion of a mechanical stop). According to some embodiments, the additional mechanism does not create a local energy minimum, but it does decrease the likelihood of spontaneous loosening by virtue of increasing the energy required to effectuate such loosening. In some embodiments, such a mechanism is used in addition to or in place of a mechanical stop 36 or another feature configured to create a local energy minimum. Thus, in some embodiments, loosening can essentially be entirely prevented absent intentional intervention or extraordinary circumstances (which would not ordinarily occur in physiological conditions).

Where a mechanism for increasing the force required to loosen the set screw 30 is included, any suitable mechanism can be used. For example, some embodiments include use of one or more: adhesives or thread-locking fluids, washers (e.g., spring washers or other washers), thread-locking nuts, threading variations to increase tightness, jam nuts, threadlocker, redundant set screws, or other components configured to assist in tightening threaded fasteners.

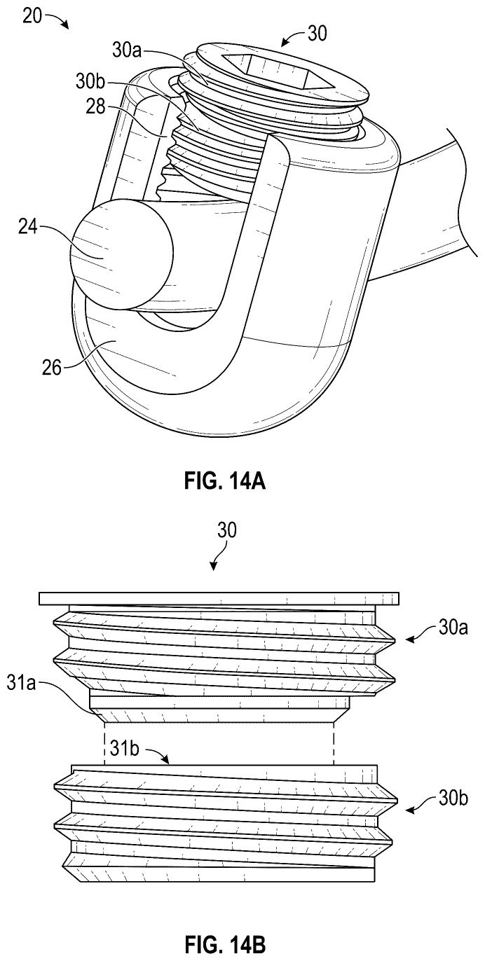

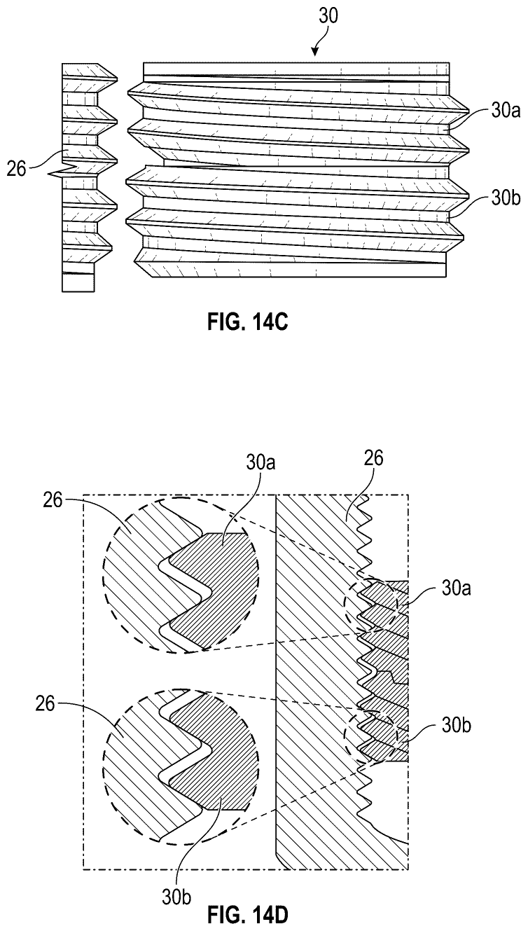

As one example, some embodiments include one or more multi-part set screws 30 configured to be inserted separately or together into the set screw seat (e.g., in the tulip 26). In some embodiments, the multi-part set screw includes a first part 30a and a second part 30b (and any number of additional parts). By way of non-limiting illustration, FIGS. 14A-14C show bifurcated set screws 30 having a first part 30a configured to insert into a second part 30b (e.g., the first part 30a has a trunk 31a configured to insert into a receptacle 31b of the second part 30b). In some embodiments, the multi-part set screw is configured to operate like a jam-nut or similar device, such that the locking strength of the set screw is greater than it would be for a single-part set screw. For example, in some cases, the interaction of the first part and the second part causes the first part to be forced upward against the threading and the second part to be forced downward against the threading, thereby created a tighter fit (e.g., as shown in FIG. 14D).

Although some embodiments of the multi-part set screw 30 are free from mechanical stops 36, in some embodiments, one or more parts of the multiple-part set screw comprise one or more mechanical stop (e.g., having any suitable feature discussed herein).

In some embodiments, multiple components of the described systems are configured to operate as jam-nuts (e.g., like the multi-part set screws in the previous paragraph, but for the tulip instead). Some embodiments include one or more additional locking or tightening features, such as one or more castellations, lock nut systems, check nut systems, sawn nut systems, ring or groove nut systems, anti-splay features, anti-backout features, split-washers, nord lock washers, serrated flanges, polymer-insert lock nuts, elastic stop nuts, lock washer, external tooth lock washer, or other such systems configured to prevent loosening.

According to some embodiments, a method of resisting set screw 30 loosening is provided. In some embodiments, the method includes providing or using any of the components discussed herein (e.g., one or more set screws having one or more mechanical stops 36, including one or more processes 38, tabs 42, pins 44, caps 52, or other types of mechanical stops) in any suitable manner. In some embodiments, the method includes one or more additional parts. For example, in some embodiments, the method includes plastically deforming the set screw or any component thereof to increase the friction of the normal force, thereby resisting set screw backout. For example, in some embodiments, during insertion the set screw is advanced at an insertion angle (e.g., 30 degrees, or any other suitable insertion angle). Once tight, the angle is plastically deformed to a retention angle (e.g., 60 degrees, or any other suitable retention angle). In some embodiments, this dramatically increases the friction forces acting on the set screw (due to the change in the angle of the normal force). The angles used (and the change of angle) can be anywhere between 5 degrees and 120 degrees, or any subrange thereof. For example, some embodiments include changing the angle by at least 10 degrees, 15 degrees, 20 degrees, 25 degrees, 30 degrees, 45 degrees, or any other suitable change for a desired force increase.

The systems and methods described herein can be modified in any suitable manner. For example, some embodiments have a selectively engageable mecahnical stop 36 that is configured to be selectively engaged or selectively disengaged using a tool, such as a general tool as may be found in an operating room or a specialized tool specifically configured to engage or disengage the mecahnical stop.

The described systems and each component disclosed herein can be made in any suitable manner, including via molding, etching, tapping, die threading, extruding, thread cutting, milling, grinding, thread rolling, cold form tapping, hot rolling, forging, pressing, stamping, casting, additive manufacturing, machining, thread whirling, ultrasonic forming, or in any other suitable manner.

In addition to the aforementioned features, the described systems and methods can include any other suitable feature. Indeed, the described systems and methods often have many advantages over existing systems. In this regard, by creating a local energy minimum, the capacity of the set screw 30 to resist loosening is far greater than for screws with no such minimum, no matter how tight such screws are initially made.

Any and all of the components in the figures, embodiments, implementations, examples, instances, cases, methods, applications, iterations, and other parts of this disclosure can be combined in any suitable manner and in any permutation. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a mechanical stop includes reference to one or more mechanical stops, and reference to tabs includes reference to one or more tabs. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The term “suitable” may refer to anything that could possibly allow the described systems and methods to function as described herein.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A spinal implant for posterior fixation comprising:
an anchor;
a rod;
a tulip comprising:
a bottom having an opening, the opening being configured to receive the anchor;
a top; and
a receptacle disposed between the bottom and the top, the receptacle being configured to receive the rod and comprising complimentary threading; and
a set screw comprising a threading configured to interface with the complementary threading of the receptacle, the set screw being configured to prevent the rod from withdrawing from the receptacle through the top of the tulip when the set screw is in place,
wherein the set screw further comprises a mechanical stop configured to resist counter-rotation of the set screw when the set screw is in place to thereby resist loosening of the set screw.

2. The spinal implant of claim 1, wherein the mechanical stop is configured to create a local energy minimum in a potential energy curve of the set screw to reduce a natural tendency of the set screw to loosen and move from a higher potential energy to a lower potential energy over time.

3. The spinal implant of claim 1, wherein at least one of the tulip and the rod comprises a corresponding stop configured to engage with the mechanical stop to resist the counter-rotation of the set screw.

4. The spinal implant of claim 1, wherein the mechanical stop comprises at least one of a process, a tab, a pin, and a cap.

5. The spinal implant of claim 1, wherein the mechanical stop comprises at least two of a process, a tab, a pin, and a cap.

6. The spinal implant of claim 1, wherein the mechanical stop comprises at least three of a process, a tab, a pin, and a cap.

7. The spinal implant of claim 1, wherein the mechanical stop comprises each of a process, a tab, a pin, and a cap.

8. The spinal implant of claim 1, wherein the tulip comprises a corresponding stop configured to interface with the mechanical stop.

9. The spinal implant of claim 1, further comprising a resistance mechanism configured to increase an amount of energy required to loosen the set screw.

10. A set screw for a spinal implant, the set screw comprising:
threading configured to interface with complementary threading of the spinal implant to allow for rotation of the set screw in a tightening direction and counter-rotation in a loosening direction; and
a mechanical stop configured to create a local energy minimum in a potential energy curve of the set screw to counteract a natural tendency of the set screw to counter-rotate from a higher potential energy state to a lower potential energy state, thereby resisting the counter-rotation of the set screw, and thereby resisting loosening of the set screw.

11. The set screw of claim 10, wherein the mechanical stop comprises at least one of a process, a tab, a pin, and a cap.

12. The set screw of claim 10, wherein the mechanical stop comprises at least two of a process, a tab, a pin, and a cap.

13. The set screw of claim 10, wherein the mechanical stop comprises at least three of a process, a tab, a pin, and a cap.

14. The set screw of claim 10, wherein the mechanical stop comprises each of a process, a tab, a pin, and a cap.

15. The set screw of claim 10, further comprising a resistance mechanism configured to increase an amount of energy required to loosen the set screw.

16. A method of providing a spinal implant having a set screw configured to resist loosening, the method comprising:
obtaining a set screw comprising threading and a mechanical stop;
obtaining an anchor;
obtaining a rod;
obtaining a tulip comprising a bottom, a top, and a receptacle;
disposing the anchor through an opening of the bottom of the tulip;
disposing the rod within the receptacle of the tulip;
rotating the set screw into the top of the tulip such that the threading of the set screw engages with a complementary threading of the tulip, thereby causing the set screw to tighten; and
engaging the mechanical stop to resist counter-rotation of the set screw, thereby resisting loosening of the set screw.

17. The method of claim 16, wherein the mechanical stop comprises at least one of a process, a tab, a pin, and a cap.

18. The method of claim 16, wherein the mechanical stop comprises at least two of a process, a tab, a pin, and a cap.

19. The method of claim 16, wherein the mechanical stop comprises at least three of a process, a tab, a pin, and a cap.

20. The method of claim 16, wherein the mechanical stop comprises each of a process, a tab, a pin, and a cap.
